(19) United States
(12) Patent Application Publication
(10) Pub. No.: US 2019/0030046 A1
(43) Pub. Date: Jan. 31, 2019

(54) USE OF PURE COMPOUNDS EXTRACTED FROM ANTRODIA CAMPHORATA

(71) Applicant: CHUN CHING SHIH, Taichung (TW)

(72) Inventor: CHUN CHING SHIH, Taichung (TW)

(21) Appl. No.: 16/073,662

(22) PCT Filed: Nov. 28, 2016
(86) PCT No.: PCT/CN2016/107285

§ 371 Date: Jul. 27, 2018

Related U.S. Application Data
(60) Provisional application No. 62/340,681, filed on May 24, 2016.

(51) Int. Cl. A61K 31/575; A61K 36/54; A61P 3/04; A61P 3/10

(52) U.S. Cl. CPC A61K 31/575; A61K 36/54; A61K 2236/33; A61P 3/10; A61P 3/04

(57) ABSTRACT

The present invention provides a use of a pure compound extracted from Antrodia camphorata in preparation of a pharmaceutical composition for controlling appetite, regulating metabolism of lipids and glucoses in peripheral tissues, alleviating a condition, or treating a disease such as metabolic syndrome, obesity, fatty liver, hyperinsulinemia, or type 2 diabetes. The pure compound comprises dehydroeburicoic acid (C₃₁H₄₈O₃, TT).

### BACKGROUND OF THE INVENTION

### 1. Technical Field

The present invention relates to a use of a pure compound extracted from Antrodia camphorata in the preparation of a pharmaceutical composition for controlling appetite, regulating metabolism of lipids and glucoses in peripheral tissues, alleviating a condition, or treating a disease such as metabolic syndrome, obesity, fatty liver, hyperinsulinemia, or type 2 diabetes. The pure compound comprises dehydroeburicoic acid (C₃₁H₄₈O₃, TT).

### 2. Description of Related Art

Diabetes mellitus is the core of multiple etiologies of chronic hyperglycemia, metabolic cardiovascular diseases and the like due to faults in insulin action or secretion or both. Among the Diabetes mellitus, type 2 diabetes affects an estimated 90-95% of all diabetes cases. The pathogenesis of type 2 diabetes includes metabolic syndrome and insulin resistance and 5% of patients with impaired β-cell function in pancreas. Insulin resistance occurs when peripheral tissues such as livers, skeletal muscles, and adipose tissues lose sensitivity to insulin so that insulin fails to exhibit its numerous biological effects, and the resulting symptomatology includes polyphagia, dyslipidemia, lipid accumulation in peripheral tissues, obesity, hyperglycemia, and hypertension. The pathology of insulin resistance is derived from heredity and life style factors. The proportion of fat in the diet often is an important cause of metabolic disorders.

The pancreas secretes insulin to maintain normal glucose homeostasis, assist glucose uptake, and modulate carbohydrate and lipid metabolism. The research data shows that insulin may cause translocation of glucose transporter 4 (GLUT4) from an intracellular location to the membrane by stimulating phosphorylation of protein kinase B (Akt) in an Akt signaling pathway, so as to promote glucose uptake by peripheral tissues such as skeletal muscle and adipose tissue. Therefore, improving expression level of membrane GLUT4 will be an approach for treating a condition of hyperglycemia; meanwhile, contents of intracellular phosphorylated Akt (phospho-Akt) and expression levels of GLUT4 protein on the membrane are both regarded as indicators for evaluating insulin sensitivity.

In addition, the research shows that facilitating peripheral glucose to enter the skeletal muscle (which is the main disposal site for glucose) may also activate the pathway via AMP-activated protein kinase (AMPK) induced by exercise, muscular contraction, or anoxia. AMPK is responsible for modulation of cell and whole body energy metabolism. Phosphorylation of the sub-unit at Thr172 is a key affecting the AMPK activity. Modulation mechanism of its phosphorylation pathway is different from that of GLUT4 translocation, but is related to metabolic disorder of lipid and glucose caused by insulin resistance. Thus, AMPK activators are expected to be favorable therapies in the treatment of diabetes and related disorders.

Metformin (Metf) is an antidiabetic drug which is commonly used in clinic. However, metformin is a low-potency compound that is used at high doses but results in only modest net efficacy, and significant side effects can also occur. Thus, it has become a focus of attention and research in medicine industry currently to develop a drug for diabetes which is more effective in clinical applications and does not cause undesired side effects.

Antrodia camphorata is a traditional Chinese medicine in Taiwan. Antrodia camphorata is rare and expensive because it only grows on the inner heartwood wall of the endemic evergreen Cinnamomum kanehirai. Antrodia camphorata has many physiologically active ingredients, including triterpenoids such as Antcin A-K, 15-acetyl-dehydrosulphurenic acid, dehydroeburicoic acid (TT), dehydrosulphurenic acid (TR4), and eburicoic acid (TR1), and the like. Antcin K (AnK) (having a chemical formula as shown in FIG. 1B) and dehydroeburicoic acid (TT) (having a chemical formula as shown in FIG. 1) are one of the most important active ingredients of the fruiting body of Antrodia camphorata.

A previous study has demonstrated when C57BL/6J mice are fed with a high-fat diet (HFD), early type 2 diabetes can be induced in the mice and the mice exhibit the following conditions, including but not limited to, markedly increased adipose weights, resistance to insulin, increase in blood glucose, and increase in serum triglyceride (TG) and total cholesterol (TC) levels.

The present invention evaluated the effect of MeOH crude extract (CruE) from Antrodia camphorata and Antcin K (AnK) on expression levels of GLUT4 and phospho-Akt in vitro, and further investigated physiological activities and mechanisms of Antcin K (AnK) and dehydroeburicoic acid (TT) on glucose lowering and lipid lowering in HFD-induced diabetic mice. The present invention also determined expression levels of target genes related to fatty acid oxidation, such as Peroxisome proliferator-activated receptor alpha (PPARα), fatty acid synthase (FAS), and carnitine palmitoyl transferase Ia (CPT-1a). Additionally, the present invention also determined expression of target genes related to glyconeogenesis, such as glucose 6-phosphoatase (G6 Pase).

### BRIEF SUMMARY OF THE INVENTION

Accordingly, an object of the present invention is to provide a use of a pure compound extracted from Antrodia camphorata, wherein the pure compound comprises dehydroeburicoic acid (C₃₁H₄₈O₃) and is used for preparing a pharmaceutical composition. The pharmaceutical composition is used for controlling appetite, regulating metabolism of lipids and glucose in peripheral tissues, and regulating or treating a series of conditions and diseases related to metabolic disorder. The disease includes but is not limited to metabolic syndrome, obesity, fatty liver, hyperinsulinemia, or type 2 diabetes.

For the purposes of this specification it will be clearly understood that the word “comprising” means “including but not limited to”, and that the word “comprises” has a corresponding meaning the same as aforesaid.

Unless defined otherwise, all technical and scientific terms used herein have the same meaning as commonly understood by one of ordinary skill in the art to which the invention belongs. Equivalents and substitutions to methods and materials described herein are possible to those skilled in the art to which the invention belongs, and can be equally used to implement the present invention. Indeed, the present invention is in no way limited to the methods and materials described herein.

According to the present invention, the pure compound further comprises Antcin K. In a particular embodiment of the present invention, Antcin K is related to physiological activity on improving hyperglycemia.

According to the present invention, the metabolic syndrome is insulin resistance syndrome. In a particular embodiment of the present invention, serum insulin level can be reduced.

According to the present invention, the condition is dyslipidemia, including TC or TG. In a particular embodiment of the present invention, elevation in free fatty acids (FFA) can be reduced.

According to the present invention, the condition is elevation in liver total lipids. In a particular embodiment of the present invention, the condition is elevation in triacylglycerol, and liver ballooning degeneration.

According to the present invention, the condition is hyperleptinemia. In a particular embodiment of the present invention, serum leptin level can be reduced.

According to the present invention, the condition is elevation in visceral fat mass caused by high fat diet. In a particular embodiment of the present invention, hypertrophy of adipocyte can be reduced.

The foregoing, and additional objects, features and advantages of the invention will become apparent to those of skill in the art from the following detailed description and preferred embodiments of the invention taken with the accompanying drawings.

### BRIEF DESCRIPTION OF THE SEVERAL VIEWS OF THE DRAWINGS

FIG. 1 is a chemical formula of dehydroeburicoic acid (TT) in a pure compound.

FIGS. 2A to 2E show the effect of administration of insulin or MeOH crude extract (CruE) from Antrodia camphorata on membrane GLUT4 and phospho-Akt/total Akt in C2C12 myotube cells in vitro, and the comparative effect of administration of insulin or dehydroeburicoic acid (TT) and Antcin K (AnK) on membrane GLUT4 and phospho-Akt/total Akt. FIGS. 2A and 2C show representative immunoblots produced in culture experiments of C2C12 myotube cells. FIGS. 2B, 2D and 2E show expression level of membrane GLUT4 protein and phospho-Akt/total Akt ratio. Myotube cells in the test of C2C12 cells are treated with MeOH crude extract or dehydroeburicoic acid (TT) or Antcin K (AnK), and equal amounts of lysates are resolved by SDS-PAGE and blotted for GLUT4, total-Akt (Ser⁴⁷³) and phospho-Akt (Ser⁴⁷³). All values are means ±SE (n=6), ^{a}p<0.001.

FIGS. 3A and 3B show pathohistological staining of epididymal white adipose (FIG. 3A) and liver tissue (FIG. 3B) of mice in the CON, HF, HF+TT1, HF+TT2, HF+TT3, HF+Feno, and HF+Metf groups via H-E staining in the animal model experiments of the present invention.

FIG. 4A shows a representative image of semiquantative RT-PCR analysis on G6 Pase, 11β-HSD1, SREBP1c, GPAT, aP2, UCP3, CPT1a, and SREBP2 mRNA levels in liver tissue of the mice in the CON, HF, HF+TT1, HF+TT2, HF+TT3, HF+Feno, and HF+Metf groups in the animal model experiments of the present invention.

FIGS. 4B to 4C show quantification of signals measured for G6 Pase, 11β-HSD1, SREBP1c, GPAT, aP2, UCP3, CPT1a, and SREBP2 by image analysis and normalization of each value through GAPDH.

FIG. 5A shows determination of skeletal muscle membrane GLUT4 and phospho-AMPK(Thr¹⁷²) protein contents of the mice in the CON, HF, HF+TT1, HF+TT2, HF+TT3, HF+Feno, and HF+Metf groups by Western blot in the animal model experiments of the present invention, and determination of phospho-AMPK(Thr¹⁷²) protein content in liver of the mice in each group as described above.

FIG. 5B shows a quantative histogram of the protein contents measured in FIG. 5A.

FIG. 6A shows determination of liver PPARα, FAS and PPARγ protein contents of the mice in the CON, HF, HF+TT1, HF+TT2, HF+TT3, HF+Feno, and HF+Metf groups by Western blot in the animal model experiments of the present invention, and determination of PPARγ and FAS protein contents in adipose tissue of the mice in each group as described above.

FIG. 6B shows a quantative histogram of the liver PPARα, FAS and PPARγ protein contents in the mice measured in FIG. 6A.

FIG. 6C shows a quantative histogram of the PPARγ and FAS protein contents in adipose tissue of the mice measured in FIG. 6A.

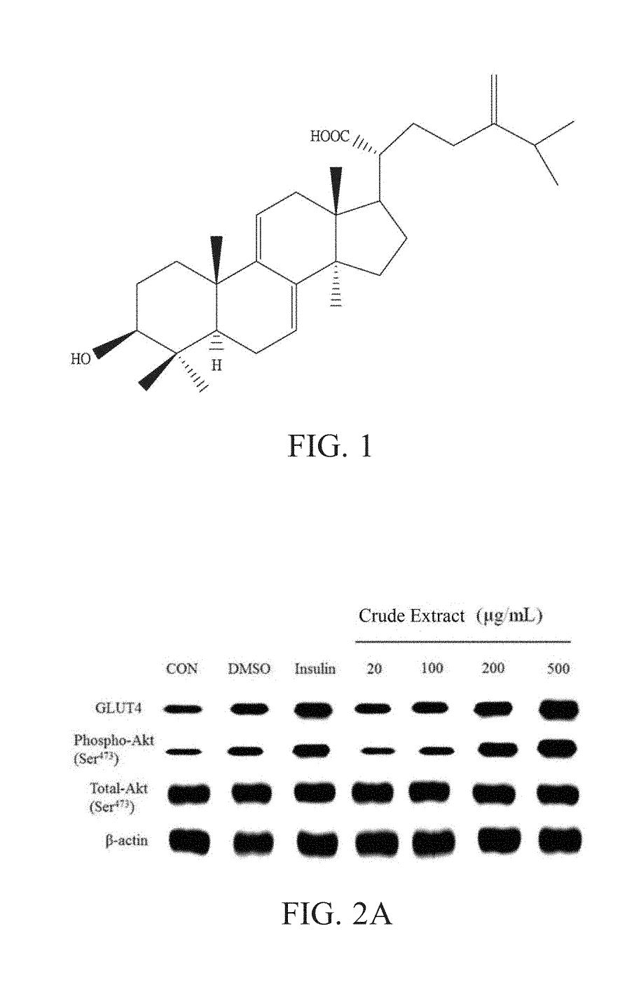

### DETAILED DESCRIPTION OF THE INVENTION

Some exemplary embodiments of the features and advantages of the present invention will be described in detail in the following description. It is to be understood that the invention can assume various changes in different aspects without departing from the scope of the present invention, and the drawings and description therein are to be regarded as illustrative in nature, and not as restrictive. The extraction method of the pure compound from Antrodia camphorata and efficiency test thereof will be further described below.

(I) Materials and Methods

Antibodies to GLUT4 (no. sc-79838) used in the present invention were purchased from Santa Cruz Biotech (Santa Cruz, Calif., USA). Phospho-AMPK (Thr172), PPARα (no. ab8934), and PPARγ (no. ab45036) antibodies were purchased from Abcam Inc. (Cambridge, Mass., USA). FAS (no. 3180), phospho-Akt (Ser473) (no. 4060), total AMPK (Thr172), and (3-actin (no. 4970) antibodies were obtained from Cell Signaling Technology (Danvers, Mass., USA). Secondary anti-rabbit antibodies were purchased from Jackson ImmunoRes. Lab., Inc. (West Grove, Pa., USA).

The mycelium of Antrodia camphorata used in the present invention was obtained from the Konald Biotech Co. Ltd. (Chiayi City, Taiwan). The extract from Antrodia camphorata used in the present invention refers on a single component extracted from a freeze-dried powder of the mycelium of Antrodia camphorata and further purified: pure compound dehydroeburicoic acid, TT). A specific extraction method of the pure compound dehydroeburicoic acid (TT) of the present invention was as follows: 3.0 kg freeze-dried powders of the mycelia of Antrodia camphorata were extracted three times with 12 L methanol (MeOH) at room temperature (4 days×3). The methanol extract of was evaporated in vacuo to yield a brown residue, which was suspended in 1 L purified water and partitioned with ethyl acetate (1 L×3). The EtOAc fraction (200 g) was chromatographed on silica gel using mixtures of hexane and EtOAc of increasing polarity as eluents and further purified with a high performance liquid chromatography (HPLC) (Shimadzu CL 20-a, Kyoto, Japan). In this example, dehydroeburicoic acid (TT) of the present invention isolated by HPLC on a Hibar pre-packed column RT 250-10 with chloroform: ethyl acetate (7:1). The flow rate was 3 mL/min, and the injection volumes of samples were 100 pt. The composition of the isolated TT was analyzed by a refractive index (RI) (Knauer RI detector 2400). The yield of the prepared TT was 0.2% (w/w), and the purity was over 99%.

Furthermore, the fruiting body of Antrodia camphorata used in the present invention was purchased from the Balay Biotechnology Corporation in Hsinchu City, Taiwan. A voucher specimen (CMPC393) was identified by China Medical University.

The fruiting bodies of AC (3.0 kg) were extracted three times with methanol followed by chromatography using 50% ethyl acetate and 50% hexane. This procedure was performed as previously reported. The purity of Antcin K(AnK) was over 99%. The analytical instruments were an HPLC (Shimadzu CL 20-A, Kyoto, Japan); the HPLC Column, TOSOH TSKgel DS-80Ts, and 100% MeOH was used as an organic solvent for washing the packed HPLC column.

1.1 Cell Culture

The following tested whether administration of insulin or MeOH crude extract (CruE) from Antrodia camphoratain C2C12 myotube cells can regulate membrane GLUT4 or affect phospho-Akt protein expression, or tested comparative effects of administration of insulin or Antcin K(AnK) or dehydroeburicoic acid (TT) to regulate membrane GLUT4 or affect phospho-Akt protein expression.

In the present invention, C2C12 skeletal myoblasts (ATCC, CRL-1772) were maintained in growth media consisting of Dulbecco's modified Eagle's medium (DMEM) (Gibco BRL) and supplemented with 10% Fetal Bovine Serum (FBS) (Hyclone) and 100 U/mL penicillin/100 μg/mL streptomycin (Gibco BRL), and split 1:4 using 0.05% trypsin when 80% confluent. Myoblasts were diluted and placed in a 9 cm-diameter dish. Cells were cultured to achieve 80%-90% confluency and then the growth media was changed as 2% FBS/DMEM every 24 h for 5-7 days.

In experiments for determining GLUT4 and p-Akt(Ser473)/t-Akt proteins in tubes, differentiated C2C12 cells were serum-starved in DMEM/BSA for 2 h at 37° C. prior to incubation with MeOH crude extract (at 20, 100, 200, and 500 μg/mL), test compounds (at 1, 5, 10, and 25 μg/mL), or vehicle for 25 min, or 100 nM insulin for 25 min.

The cultures obtained by cell culture above were centrifuged, and the resulting pellet was resuspended in a homogenization buffer. The forgoing steps were performed with a filtration membrane. Protein concentrations were measured using a BCA assay (Pierce). Equal amounts of protein were diluted four times in SDS sample buffer, and subjected to SDS PAGE and Western blotting with antibodies specific for Akt, p-Akt Ser⁴⁷³, and GLUT4. Density blotting analysis was performed using Alpha Easy FC software.

1.2 Animal Model Experiment

Fenofibrate (Feno) is a PPARα activator and has been used in treatment of hypertriglyceridemia for many years. PPARα is a key regulating factor of lipid metabolism ralated genes, and leads to decrease in blood triglyceride and fatty acid by participating in the regulation of many target genes such as lipid production, fatty acid oxidation, and energy consumption.

Metformin is an antidiabetic drug currently widely used for treatment of type 2 diabetes. It can activate AMPK in liver and skeletal muscle.

Thus, fenofibrate and metformin were selected as control drugs for evaluating the therapeutic effect of dehydroeburicoic acid (TT) against type 2 diabetes. To determine whether the performance of GLUT4 and phospho-AMPK in regulating blood glucose in vivo changes and whether phosphorylation of Thr172 is essential for AMPK activity, the present invention used HFD-induced diabetic mice as an animal model of type 2 diabetes and detected skeletal muscle and liver tissue of the mice, whereby the effect of TT against type 2 diabetes and against dyslipidemia was investigated and performance of TT against type 2 diabetes and lipogenesis and in regulating target genes including PPARα and FAS in liver tissue was determined.

A design of animal model experiments of the present invention for testing the efficacy of the pure compound dehydroeburicoic acid (TT) of the extract from Antrodia camphorata in preventing or treating type 2 diabetes and dyslipidemia, and blood data, serum biochemical analysis, histopathological analysis, liver lipid analysis, and RNA extraction and relative quantification of mRNA target gene expression are explained below.

4-week old male C57BL/6J mice (63 in total) were purchased from the National Laboratory Animal Breeding Center, and were divided into a control/low-fat-diet group (low-fat-diet; CON (CD; Diet 12450B, Research Diets, Inc., New Brunswick, N.J., USA) (9 in total) and a high-fat-diet group (HFD (Diet 12451, Research Diets, Inc.) (54 in total). The diet of CON was composed of 20% protein, 70% carbohydrate and 10% fat. The diet of HFD was composed of 20% protein, 35% carbohydrate and 45% fat. The two groups of experimental mice were fed with distinct diets for 12 weeks in the experiment of the present invention.

The control group and HFD group contained 10% and 45% fat intake, respectively. The HFD mice were subdivided into 6 groups (9 per group) followed by 8-week induction. The 6 groups of the mice were further administered with four-week treatment with the pure compound or drugs, including 6 different types and doses of the drugs: (1) TT1=dehydroeburicoic acid 10 mg/kg/day, (2) TT2=dehydroeburicoic acid 20 mg/kg/day, (3) TT3=dehydroeburicoic acid 40 mg/kg/day, (4) Feno (Sigma Chemical Co., St Louis, Mo., USA)=fenofibrate 0.25 g/kg/day, (5) Metf (Sigma Chemical Co., St Louis, Mo., USA)=metformin 0.3 g/kg/day, and (6) an equivalent dose of distilled water (vehicle). The distilled water, ehydroeburicoic acid (TT), metformin, or fenofibrate was orally administered via oral gavage once daily for the last 28 days (four weeks). During the experiment, all mice were fasted overnight, and blood samples were collected from the retro-orbital sinus. Following four-week treatment and feeding, the food was removed, and the mice were fasted for 12 h and then sacrificed. Tissue samples required were collected from the mice and weighted. Some of the tissue samples were immediately stored at −80° C. and frozen for subsequent target gene analysis. Plasma samples were collected via centrifugation of whole blood at 1600×g for 15 min at 4° C., followed by plasma separation within 30 min. Some plasma samples obtained were used for TG and TC analysis. The metabolic parameters, including body weight, weight gain, and food intake, were performed as follows. Body weight was daily measured throughout the study. Body weight gain is considered as the difference between one day and the next day. The total weight of food intake was daily measured, followed by weighing the amount of remaining feed after 24 h. The difference indicates the daily food intake.

1.3 Measurement of Fasting Blood Glucose and Biochemical Indexes

One part of the forgoing blood samples collected from the retro-orbital sinus of 12 h fasted mice was immediately used for analysis of glucose levels. The other part was used for analysis of blood TG, TC, free fatty acids, blood insulin, leptin, and adiponectin levels. Blood glucose levels were determined by a glucose analyzer (Model 1500 sidekick glucose analyzer, YSI). Plasma triglycerides (TG), total cholesterol (TC), and free fatty acids (FFA) were determined using commercial kits in accordance with manufacturer's instructions (Triglycerides-E test, Cholesterol-E test, and FFA-C test; Wako Pure Chemical, Osaka, Japan). Insulin and leptin levels were measured by enzyme-linked immunosorbent assay (ELISA) (mouse insulin ELISA kit, Mercodia, Uppsala, Sweden; mouse leptin ELISA kit, Morinaga, Yokohama, Japan).

1.4 Histopathological Analysis

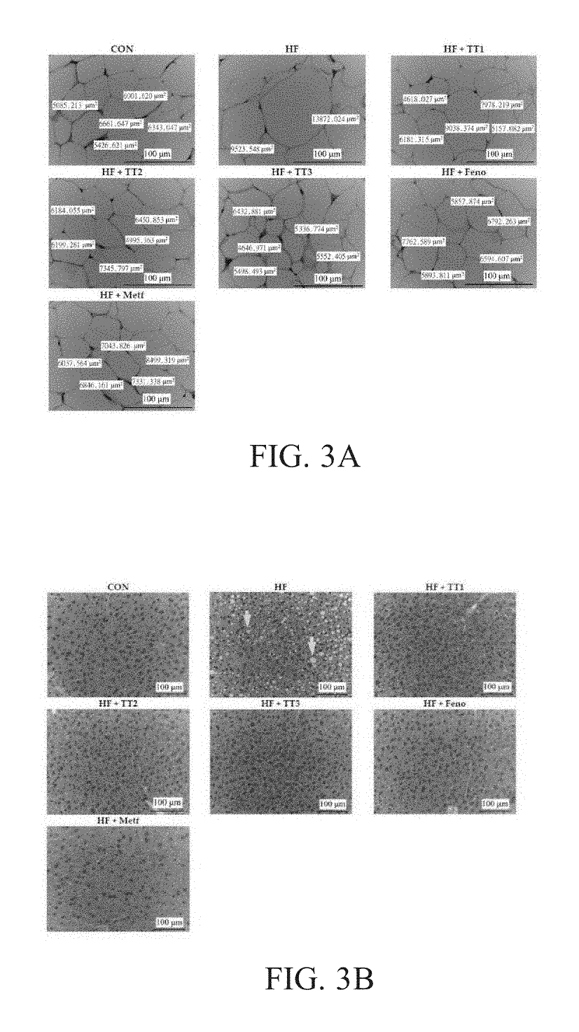

Examination was performed for parts of EWAT (epididymal white adipose) and liver tissue specimens collected. The specimens above were soaked in formalin and neutral buffered solution and embedded in paraffin. Parts therein (8 μm) were cut and stained with hematoxylin and eosin. Images were photographed by a microscope (Olympus BX51, Olympus, Tokyo, Japan). Please refer to FIGS. 3(A) and 3(B).

Analysis of Liver Lipids

Hepatic lipase was analyzed in accordance with previous procedures. The extracted hepatic lipase samples (0.375 g) were homogenized with distill water (1 mL) for 5 min. Finally, the dried pellet was resuspended in ethanol (0.5 mL) and analyzed using a triglycerides kit as blood triglycerides kit.

1.5 Gene Expression by Relative Quantization of mRNA and Western Blotting

Total RNA from liver tissue was isolated with a Trizol Reagent (Molecular Research Center, Inc., Cincinnati, Ohio, USA) according to the manufacturer's instructions. The integrity of the extracted total RNA was examined by 2% agarose gel electrophoresis, and the RNA concentration was determined by 2% agarose gel electrophoresis and the ultraviolet (UV) light absorbency at 260 nm and 280 nm (Spectrophotometer U-2800A, Hitachi). The total RNA (1 μg) was reverse transcribed to cDNA, and 5 μL Moloney murine leukemia virus reverse transcriptase (TEpicentre, Madison, Wis., USA). The PCR was performed in a final 25 μL containing 1 U Blend Taq-Plus (TOYOBO, Japan), 10 μL of the RT cDNA product, 10 μM of each forward (F) and reverse (R) primer, 75 mM Tris-HCL (pH 8.3) containing 1 mg/L Tween 20 (pH 8.3, also known as polyoxyethylene sorbitan monolaurate), 2.5 mM dNTP (deoxy-ribonucleoside triphosphate), and 2 mM MgCl2 (magnesium chloride). The primers used are shown in Table 1 below. The PCR products were analyzed by 2% agarose gel and stained with ethidium bromide.

TABLE 1
List of primers used in the present invention

| | | | | |
|---|---|---|---|---|
| | | | PCR | Annealing |
| | Accession | Forward primer & | product | temperature |
| Gene | number | Reverse primer | (bp) | (° C.) |

Liver tissue

| | | | | |
|---|---|---|---|---|
| G6 Pase | NM_008061.3 | F:GAACAACTAAAGCCTCTGAAAC | 350 | 50 |
| | | R:TTGCTCGATACATAAAACACTC | | |
| SREBP1c | NM_011480 | F:GGCTGTTGTCTACCATAAGC | 219 | 48 |
| | | R:AGGAAGAAACGTGTCAAGAA | | |
| GPAT | BC019201.1 | F:CAGTCCTGAATAAGAGGT | 441 | 51 |
| | | R:TGGACAAAGATGGCAGCAGA | | |
| apo C-III | NM_023114.3 | F:CAGTTTTATCCCTAGAAGCA | 349 | 47 |
| | | R:TCTCACGACTCAATAGCTG | | |
| CPT1a | BC054791.1 | F:CTTGTGACCCTACTACATCC | 332 | 51 |
| | | R:TCATAGCAGAACCTTAATCC | | |
| SREBP2 | AF289715.2 | F:ATATCATTGAAAAGCGCTAC | 256 | 48 |
| | | R:ATTTTCAAGTCCACATCACT | | |
| aP2 | NM_024406 | F:TCACCTGGAAGACAGCTCCT | 142 | 52 |
| | | R:TGCCTGCCACTTTCCTTGT | | |
| UCP3 | NM_009464 | F:GAGGTGACTACAGCCTTCTG | 242 | 51 |
| | | R:TAGGAAGTGCTTCCATGTCT | | |
| 11β-HSD1 | NM_008288.2 | F:AAGCAGAGCAATGGCAGCAT | 300 | 50 |
| | | R:GAGCAATCATAGGCTGGGTCA | | |
| β-actin | NM_007392 | F:TCTCCACCTTCCAGCAGATGT | 92 | 60 |
| | | R:AGCTCAGTAACAGTCCGCCTAGA | | |

Immunoblot method was used for the measurement of muscular membrane GLUT4 and hepatic and muscular p-AMPK(Thr¹⁷²). We determined PPARα and FAS protein expression levels in liver tissue, PPARγ and FAS protein expression levels in adipose tissue, and membrane GLUT4 expression in skeletal muscle adipose. The total membrane fraction was collected with the buffer and determined as previously described. The membrane GLUT4, p-AMPK, and total AMPK protein content were determined by Western Blotting as previously described.

(II) Test Results

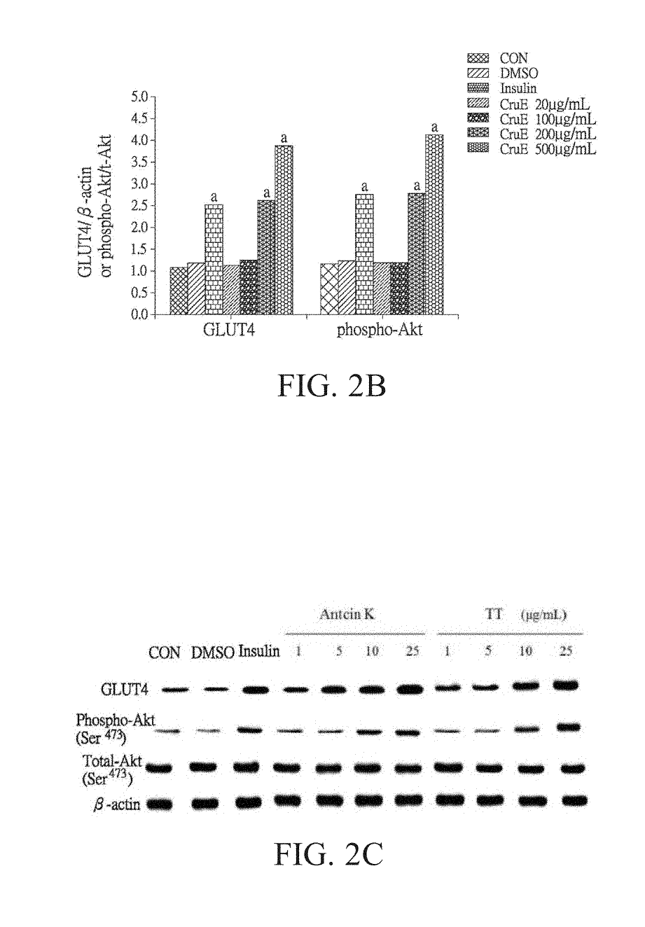

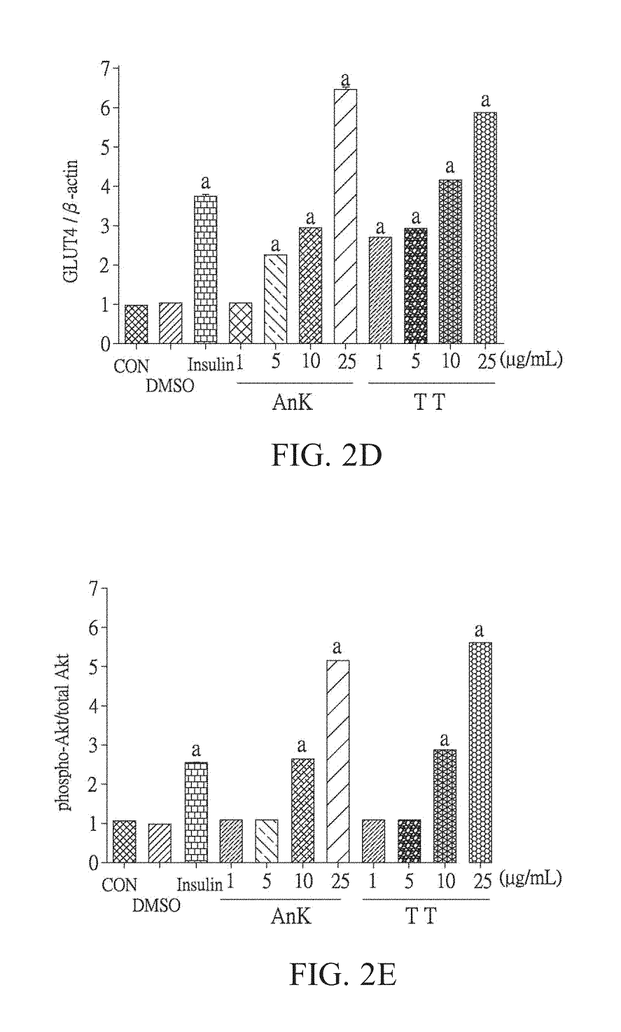

The test results of cell culture of the present invention. The experimental results show that administration of insulin, CruE (200, 500 μg/mL) in C2C12 myoblasts can increase the protein expression levels of membrane GLUT4 and phospho-Akt(Ser⁴⁷³)/total-Akt(Ser⁴⁷³) (FIGS. 2A, 2B). Administration of insulin, AnK (5, 10, and 25 μg/mL), and TT (1, 5, 10, and 25 μg/mL) can increase the protein expression level of membrane GLUT4. Administration of insulin, AnK (10 and 25 μg/mL), and TT (10 and 25 μg/mL) can increase the protein expression level of phospho-Akt(Ser⁴⁷³)/total-Akt(Ser⁴⁷³) (FIGS. 2C, 2D, and 2E). It is shown in the MTT(3-(4,5-dimethylthiazol-2-yl)-2,5-diphenyltetrazolium bromide) test that, administration of TT (at a dose of 1 to 25 μg/mL) is non-toxic to C2C12 myoblasts.

The test results in mice with type 2 diabetes induced with high-fat-diet in the animal model experiment of the present invention are explained below in conjunction with Table 2. In the animal model experiment, the effect of administration of dehydroeburicoic acid (TT) on absolute weight of tissue, intake and blood parameters in HFD-induced diabetic mice (Table 2).

The histopathological effect of administration of dehydroeburicoic acid (TT) in epididymal white adipose/liver tissue (FIGS. 3A and 3B).

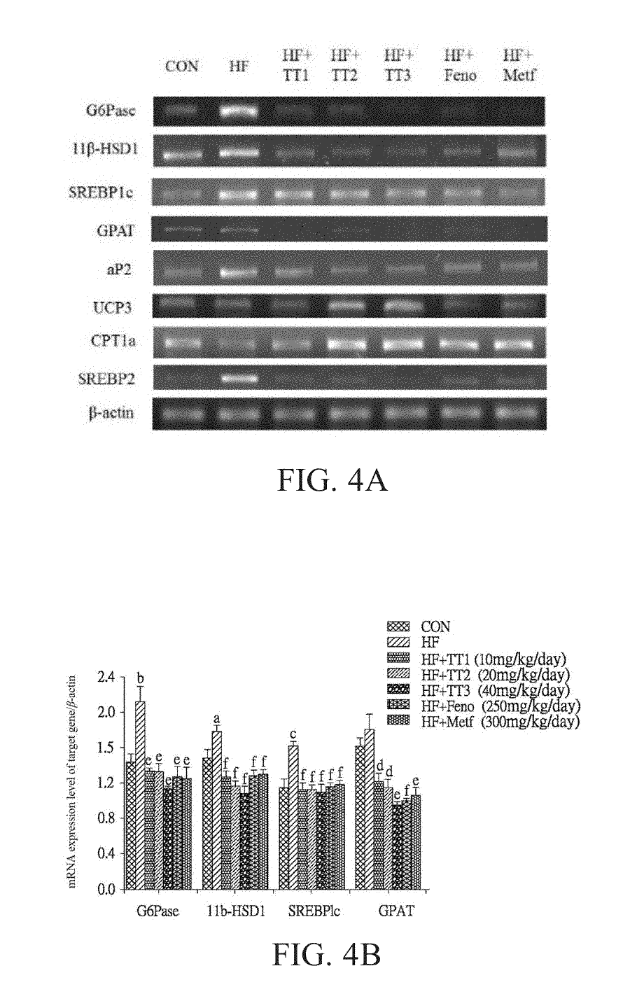

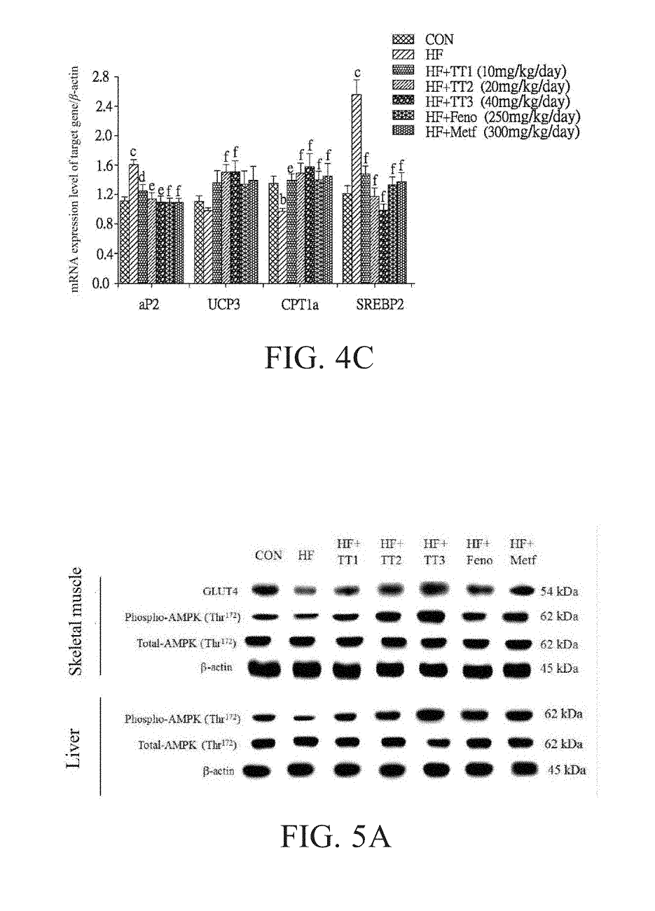

The effect of administration of dehydroeburicoic acid (TT) on semiquantative RT-PCR of G6-Pase, 11beta-HSD1, SREBP1c, GPAT, aP2, UCP3, CPT1a, SREBP-2, and beta-actin expression in liver tissue of HFD-induced diabetic mice (FIG. 4A, 4B, and 4C).

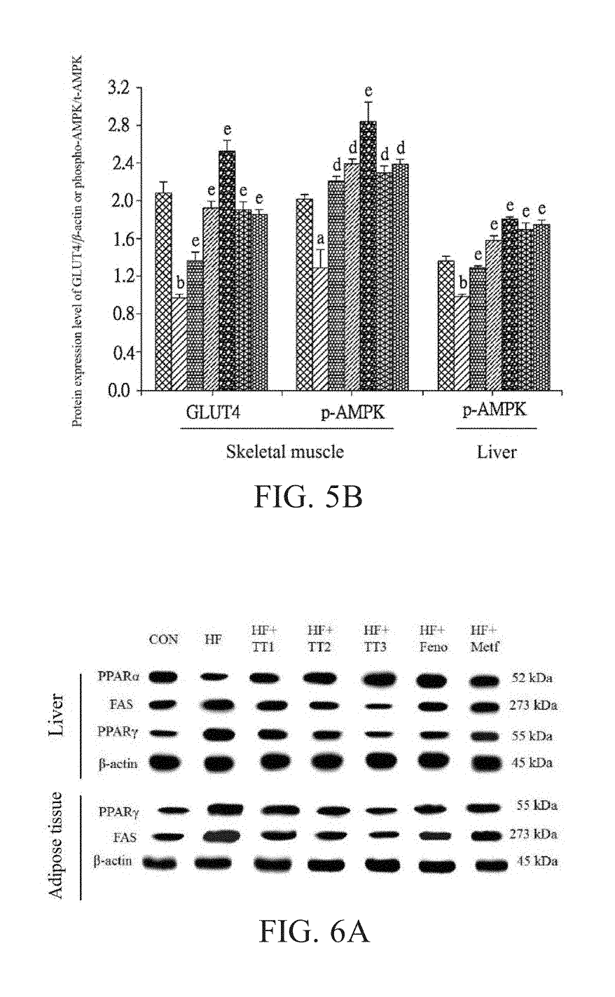

The effect of administration of dehydroeburicoic acid (TT) on measurement of the protein expression levels of membrane GLUT4, phospho-AMPK(Thr¹⁷²), total-AMPK(Thr¹⁷²), GAPDH in skeletal muscle; and phospho-AMPK(Thr¹⁷²), total-AMPK(Thr¹⁷², β-actin, and GAPDH in liver in HFD-induced diabetic mice (FIGS. 5A and 5B).

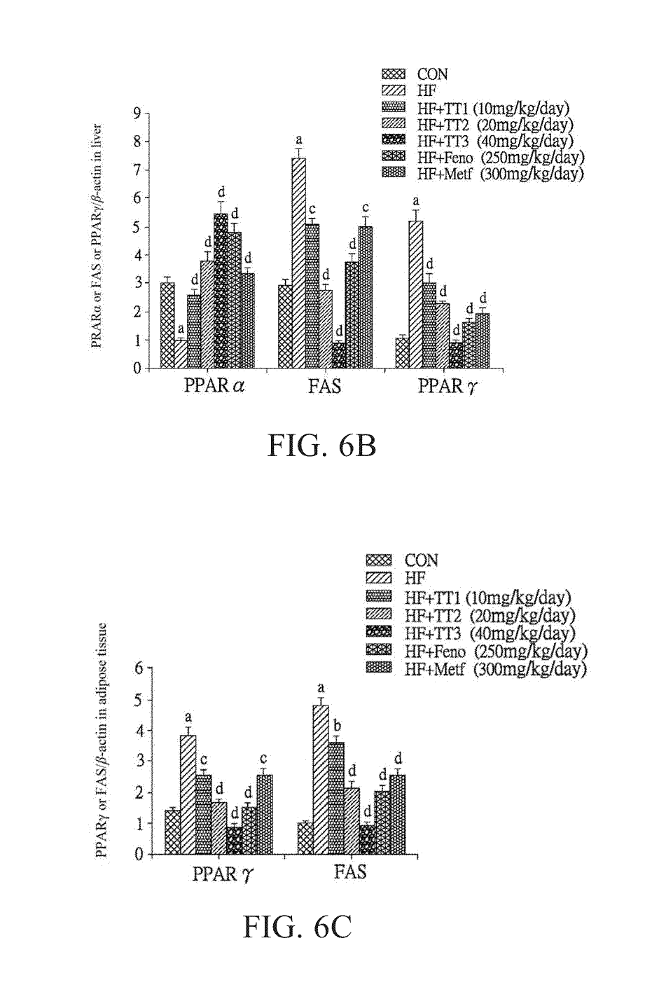

Measurement of the protein contents of PPARα, FAS, PPARγ, and β-actin in liver (FIGS. 6A, 6B, and 6C); PPARγ, FAS, and β-actin in adipocytes in HFD-induced diabetic mice (FIGS. 6A, 6B, and 6C).

2.1 Expression Levels of Membrane GLUT4 Protein and Phospho-Akt In Vitro

Referring to FIGS. 2A and 2B, analysis results of protein expression levels in C2C12 skeletal myoblasts after cell culture with different culture media in the cell culture experiment of the present invention are shown. CON (control) represents a blank control group cultured with DMEM (hereinafter referred to as CON group), DMSO represents a control group cultured with DMSO (hereinafter referred to as DMSO group), and Insulin represents an experiment group cultured with insulin (hereinafter referred to as Insulin group). Protein contents of membrane GLUT4, phospho-Akt(Ser⁴⁷³), total-Akt(Ser⁴⁷³), and β-actin in the CON, DMSO, Insulin, and CruE groups (20, 100, 200, and 500 μg/mL) (FIGS. 2A and 2B). Protein levels of membrane GLUT4, phospho-Akt (Ser⁴⁷³), total-Akt (Ser⁴⁷³), and β-actin in the CON, DMSO, Insulin, and AnK groups (1, 5, 10, and 25 μg/mL), and TT group (1, 5, 10, and 25 μg/mL) (FIGS. 2C, 2D, and 2E). In FIGS. 2A to 2E, a is indicative of statistical analysis results compared to the CON group, and indicates ^{a}P<0.001.

2.2 Analysis of Metabolic Parameters

Please referring to Table 2, analysis results of tissue and blood levels in HFD-induced mice with type 2 diabetes when fed with various doses of pure compound dehydroeburicoic acid (TT), Metformin, or Fenofibrate in the animal model experiment of the present invention the are shown. CON (control) represents a blank control group (hereinafter referred to as CON group), HFD represents a control group induced with HFD (hereinafter referred to as HF group), HF+TT1, HF+TT2, and HF+TT3 represent an experiment groups administrated with different doses of pure compound dehydroeburicoic acid (TT), respectively (hereinafter referred to as HF+TT1 group, HF+TT2 group, and HF+TT3 group), HF+Metf represents an experiment group induced with HFD and orally administered with drug Metformin (Metf) (hereinafter referred to as HF+Metf group), and HF+Feno represents an experiment group induced with HFD and orally administered with drug Fenofibrate (Feno) (hereinafter referred to as HF+Feno group). The superscripts a, b, c are indicative of statistical analysis results compared to the CON group, where ^{a}P<0.05, ^{b}P<0.01, and ^{c}P<0.001. The superscripts d, e, f are indicative of statistical analysis results compared to the HF+water (vehicle) group, where ^{d}P<0.05, ^{e}P<0.01, and ^{f}P<0.001.

TABLE 2
Effect of dehydroeburicoic acid (TT) on absolute tissue weight,
food intake, hepatic lipase, and blood parameters.

| Administration | Control | High-fat-diet | HF + | HF + | HF + | HF + | HF + |
|---|---|---|---|---|---|---|---|
| dose | group | group | TT1 | TT2 | TT3 | Feno | Metf |
| (mg/kg/day) | CON) | (HF) | 10 | 20 | 40 | 250 | 300 |

Absolute tissue weight (g)

| | | | | | | | |
|---|---|---|---|---|---|---|---|
| Epididymal | 423.6 ± | 963.1 ± | 798.9 ± | 784.3 ± | 667.8 ± | 663.1 ± | 482.1 ± |
| white adipose | 22.4 | 68.7^{c} | 43.2^{c} | 102.0^{b} | 53.2^{c,e} | 47.0^{b,e} | 31.5^{f} |
| tissue (EWAT) | | | | | | | |
| Mesenteric | 246.3 ± | 369.5 ± | 361.3 ± | 340.9 ± | 304.6 ± | 275.9 ± | 198.2 ± |
| white adipose | 7.3 | 24.2^{b} | 41.1^{a} | 28.4 | 29.5 | 26.1^{d} | 24.2^{f} |
| tissue (MWAT) | | | | | | | |
| Retroperitoneal | 133.5 ± | 470.4 ± | 343.9 ± | 331.9 ± | 311.4 ± | 268.6 ± | 180.4 ± |
| white adipose | 11.8 | 45.6^{c} | 23.4^{c} | 47.5^{c} | 33.6^{c,e} | 26.2^{b,e} | 15.7^{f} |
| tissue (RWAT) | | | | | | | |
| Visceral fat | 557.0 ± | 1433.4 ± | 1142.9 ± | 1116.2 ± | 979.3 ± | 931.7 ± | 662.4 ± |
| | 31.5 | 112.9^{c} | 32.4^{b} | 131.5^{c,d} | 86.2^{c,e} | 70.6^{b,e} | 44.0^{f} |
| Liver(g) | 878.5 ± | 889.0 ± | 848.3 ± | 841.8 ± | 802.8 ± | 882.4 ± | 1959.0 ± |
| | 28.0 | 15.3 | 30.6^{d} | 13.4^{a,d} | 24.4^{d} | 24.1 | 105.6^{c,f} |
| Spleen(g) | 76.0 ± | 86.1 ± | 77.7 ± | 81.5 ± | 82.3 ± | 74.6 ± | 81.0 ± |
| | 2.6 | 1.7 | 3.0 | 10.1 | 3.7 | 4.5 | 3.5 |
| Brown adipose | 135.6 ± | 249.0 ± | 228.6 ± | 197.3 ± | 198.1 ± | 170.4 ± | 134.2 ± |
| tissue (BAT) | 9.7 | 15.3^{c} | 23.9^{c} | 12.9^{c,a} | 14.2^{c,a} | 11.0^{a,e} | 5.1^{f} |
| Weight gain | 1.56 ± | 2.89 ± | 2.24 ± | 0.89 ± | 0.83 ± | 0.46 ± | 0.52 ± |
| | 0.19 | 0.27^{c} | 0.14^{a,d} | 0.26^{f} | 0.21^{a,f} | 0.24^{b,f} | 0.29^{a,f} |
| Final body | 24.72 ± | 28.55 ± | 27.52 ± | 26.47 ± | 26.53 ± | 26.04 ± | 26.35 ± |
| weight | 0.48 | 0.64^{c} | 0.67^{b} | 0.57^{d} | 0.91^{d} | 0.60^{d} | 0.85^{d} |
| Food intake | 2.45 ± | 2.26 ± | 2.21 ± | 2.16 ± | 2.07 ± | 2.05 ± | 2.25 ± |
| (g/day/mouse) | 0.05 | 0.06^{a} | 0.05^{c} | 0.05^{c} | 0.04^{c,d} | 0.06^{c,d} | 0.06^{a} |

Liver lipids

| | | | | | | | |
|---|---|---|---|---|---|---|---|
| Total lipid | 56.9 ± | 89.7 ± | 64.4 ± | 61.7 ± | 57.9 ± | 62.0 ± | 61.1 ± |
| (mg/g) | 1.4 | 2.1^{c} | 1.8^{b,f} | 1.9^{f} | 1.4^{f} | 2.1^{f} | 1.9^{f} |
| Triacylglycerol | 45.9 ± | 78.4 ± | 53.0 ± | 48.0 ± | 47.1 ± | 48.6 ± | 49.0 ± |
| (mol/g) | 3.0 | 4.6^{c} | 2.6^{f} | 2.4^{f} | 2.0^{f} | 2.0^{f} | 2.1^{f} |

Blood parameters

| | | | | | | | |
|---|---|---|---|---|---|---|---|
| Plasma FFA | 0.96 ± | 1.28 ± | 0.95 ± | 0.87 ± | 0.81 ± | 0.86 ± | 0.85 ± |
| (meq/L) | 0.13 | 0.19^{c} | 0.09^{f} | 0.120^{f} | 0.06^{b,f} | 0.08^{a,f} | 0.09^{a,f} |
| Blood | 78.56 ± | 137.44 ± | 90.44 ± | 82.89 ± | 77.78 ± | 86.56 ± | 87.33 ± |
| glucose(mg/dL) | 1.71 | 2.62^{c} | 1.80^{c,f} | 1.91^{f} | 2.44^{f} | 3.23^{a,f} | 2.78^{a,f} |
| TG (mg/dL) | 83.82 ± | 105.27 ± | 84.85 ± | 83.63 ± | 82.31 ± | 83.35 ± | 82.07 ± |
| | 2.14 | 1.10^{c} | 1.36^{f} | 2.42^{f} | 1.60^{f} | 2.09^{f} | 1.53^{f} |
| TC (mg/dL) | 101.70 ± | 152.71 ± | 120.55 ± | 114.93 ± | 112.46 ± | 115.25 ± | 117.20 ± |
| | 1.17 | 4.15^{c} | 0.92^{c,f} | 2.74^{c,f} | 1.76^{c,f} | 3.05^{c,f} | 3.16^{c,f} |
| Insulin (ug/L) | 2.247 ± | 3.563 ± | 2.982 ± | 2.625 ± | 2.190 ± | 2.402 ± | 2.467 ± |
| | 0.010 | 0.004^{c} | 0.024^{b,f} | 0.017^{b,f} | 0.025^{f} | 0.029^{b,f} | 0.025^{b,f} |
| Leptin (ng/mL) | 6.640 ± | 14.116 ± | 9.827 ± | 8.468 ± | 6.439 ± | 7.507 ± | 7.359 ± |
| | 0.168 | 0.244^{c} | 0.052^{c,f} | 0.182^{c,f} | 0.091^{f} | 0.144^{b,f} | 0.1325^{b,f} |

From the data results in Table 2, at the end of the experiment, final body weights and body weight gains of all high-fat fed mice are significant relative to the CON group; by comparing the groups treated with the drugs, final body weights of the mice in the HF+TT2, HF+TT3, HF+Feno, and HF+Metf groups are significantly decreased (relative to the HF group). Additionally, body weight gains of the mice in the HF+TT1, HF+TT2, HF+TT3, HF+Feno, and HF+Metf groups are also significantly decreased (relative to the HF group). Food intakes in experimental mice of HF are lower than those in CON, but food intakes in HF+TT3 and HF+Feno are lower than those in HF. For feeding with HFD, absolute weights of Epididymal white adipose tissue (EWAT), Mesenteric white adipose tissue (MWAT), Retroperitoneal white adipose tissue (RWAT), and visceral fat and brown adipose tissue (BAT) are increased relative to CON. After administration with dehydroeburicoic acid, the weights of epididymal white adipose (EWAT) and Retroperitoneal white adipose tissue (RWAT) in HF+TT3, HF+Feno and HF+Metf are significantly decreased relative to HF. Visceral fat represented epididymal WAT (EWAT) plus retroperitoneal WAT (RWAT). After administration with dehydroeburicoic acid, the weights of visceral fat and brown adipose tissue in HF+TT2, HF+TT3, HF+Feno, and HF+Metf are significantly decreased relative to HF. After administration with dehydroeburicoic acid, the weights of liver tissue in HF+TT1, HF+TT2, and HF+TT3 are decreased relative to HF, but the weights of liver tissue in HF+Metf are significantly increased.

2.3 Analysis of Blood Glucose, Insulin, and Leptin Indexes

From data in Table 2, the mice with type 2 diabetes induced with high-fat-diet exhibit increased blood glucose, insulin, and leptin compared to CON. The mice in HF+TT1, HF+TT2, HF+TT3, and HF+Feno and HF+Metf fed with dehydroeburicoic acid or two control drugs exhibit greatly decreased blood glucose levels compared to HF. The mice in HF+TT1, HF+TT2, and HF+Feno and HF+Metf fed with dehydroeburicoic acid or two control drugs exhibit greatly decreased blood insulin and leptin levels compared to HF, but the experimental mice in HF+TT3 exhibit the same levels as CON.

2.4 Analysis of Blood Triglycerides, Total Cholesterol, and Hepatic Lipase Indexes

From data in Table 2, the experimental mice with type 2 diabetes induced with high-fat-diet exhibit higher plasma triglycerides (TG), total cholesterol (TC), and free fatty acids (FFA) compared to CON. The mice in HF+TT1, HF+TT2, HF+TT3, and HF+Feno and HF+Metf fed with dehydroeburicoic acid or two control drugs exhibit decreased TG, TC, and FFA level indexes compared to HF. The experimental mice with type 2 diabetes induced with high-fat-diet exhibit increased liver total lipids and liver triglycerides compared to CON. The mice in HF+TT1, HF+TT2, HF+TT3, and HF+Feno and HF+Metf fed with dehydroeburicoic acid or two control drugs exhibit decreased liver total lipids and liver triglycerides compared to HF.

2.5 Pathohistological Examination

With 12-week high-fat-diet induction, the mice in the HF group exhibit hypertrophy of adipocyte compared to those in CON (in the experiment, adipocyte area of HF mice: 11212.6±485.2 μm²; CON mice: 6033.1±258.8 μm²), to give the data in a table below: (also refer to FIG. 3A).

| Experimental group | Adipocyte area (μm²) |
|---|---|
| HF + TT1 group | 6872.5 ± 160.8 |
| HF + TT2 group | 6530.2 ± 148.2 |
| HF + TT3 group | 5972.4 ± 279.5 |
| HF + Feno group | 6270.5 ± 165.4 |
| HF + Metf group | 6919.5 ± 195.1 |

Hight-fat-diet induction leads to significant ballooning degeneration in liver cells. This study has found that ballooning degeneration of liver cells occurs in the HF mice, resulting in hepatocellular death, hepatic glycogen accumulates among cells, and in FIG. 3, nucleoli are thus pressed to the other side, which situation is called liver ballooning degeneration (shown by the arrow in the figure).

2.6 Target Gene Expression Levels in Liver Tissue

As shown in FIGS. 4A, 4B, and 4C, G6-Pase, 11beta-HSD1, SREBP-1c, aP2, and SREBP-2 in HF have higher mRNA expression levels compared to CON, but CPT-1a has lower mRNA expression level compared to CON. After feeding with dehydroeburicoic acid or two control drugs, HF+TT1, HF+TT2, HF+TT3, HF+Feno, and HF+Metf show decreased mRNA expression levels of G6-Pase, 11β-HSD1, SREBP-1c, aP2, GPAT, and SREBP-2, but mRNA expression level of CPT-1a is increased, where mRNA expression levels of UCP3 in HF+TT2 and HF+TT3 are increased.

2.7 Target Gene Expression Levels in Various Tissues

As shown in FIGS. 5A and 5B, at the end of the experiment, membrane expressions levels of GLUT4 in skeletal muscle in HF are lower than that in CON (statistical analysis results: P<0.01). After feeding with dehydroeburicoic acid or two control drugs, membrane expressions levels of GLUT4 in skeletal muscle in HF+TT1, HF+TT2, HF+TT3, HF+Feno, and HF+Metf are significantly increased compared to HF (statistical analysis results: P<0.001, P<0.001, P<0.001, P<0.001, and P<0.001, respectively). Protein expressions levels of phospho-AMPK/total -AMPK in skeletal muscle and liver in HF are lower than CON (statistical analysis results: P<0.05 and P<0.01, respectively). After feeding with dehydroeburicoic acid or two control drugs, membrane expressions levels of phospho-AMPK/total-AMPK in skeletal muscle in HF+TT1, HF+TT2, HF+TT3, HF+Feno, and HF+Metf are significantly increased compared to HF (statistical analysis results: P<0.01, P<0.01, P<0.001, P<0.01, and P<0.01, respectively). After feeding with dehydroeburicoic acid or two control drugs, expressions levels of p-AMPK/t-AMPK proteins in liver in HF+TT1, HF+TT2, HF+TT3, HF+Feno, and HF+Metf are significantly increased compared to HF (statistical analysis results: P<0.001, P<0.001, P<0.001, P<0.001, and P<0.001, respectively).

As shown in FIGS. 6A, 6B, and 6C, PPARα protein expression level in liver in HF are lower than that in CON (statistical analysis results: P<0.001). After feeding with dehydroeburicoic acid or two control drugs, PPAR protein expressions level in liver in HF+TT1, HF+TT2, HF+TT3, HF+Feno, and HF+Metf is significantly increased compared to HF (statistical analysis results: P<0.001, P<0.001, P<0.001, P<0.001, and P<0.001, respectively). FAS and PPARγ protein expression levels in liver in HF are higher than those in CON (statistical analysis results: P<0.001 and P<0.001, respectively). After feeding with dehydroeburicoic acid or two control drugs, FAS and PPARγ protein expression levels in liver in HF+TT1, HF+TT2, HF+TT3, HF+Feno, and HF+Metf are significantly decreased compared to HF. Furthermore, PPARγ and FAS protein expression levels in adipose tissue in HF are higher than those in CON (statistical analysis results: P<0.001 and P<0.001, respectively). Likewise, after feeding with dehydroeburicoic acid or two control drugs, PPARγ and FAS protein expression levels in adipose tissue in HF+TT1, HF+TT2, HF+TT3, HF+Feno, and HF+Metf are significantly decreased compared to HF.

In summary, through the test results of cell culture of C2C12 myoblasts, the present applicant has found that Antcin K (AnK) and dehydroeburicoic acid (TT) can increase protein expression levels of membrane GLUT4 and phospho-Akt (Ser⁴⁷³), which shows that AnK and TT have positive effects on glucose uptake by cells in skeletal muscle. The expression results of target genes show that TT can reduce mRNA expression level of G6-Pase, achieving beneficial effect in inhibiting liver glucose production and attenuating diabetic conditions such as hyperglycemia.

Through pathohistological staining and animal model experiment, it has been shown that TT can reduce body weight gain, elevation in visceral fat, and liver ballooning degeneration, and animal model experiment and expression results of target genes show that TT achieves the effect of reducing lipogenesis by decreasing blood leptin level and increasing liver protein expression level of PPARα and mRNA expression level of CPT-1a.

Through animal model experiment, it has been shown that TT can reduce elevation in plasma free fatty acid, and elevation in triacylglycerol and total cholesterol, and expression results of target genes show that TT achieves the effect of promoting reduction of blood triacylglycerol by decreasing FAS protein expression level and decreasing mRNA expression levels of SREBP1c, aP2, and GPAT.

In addition, animal model experiment shows that TT can also reduce blood insulin level, facilitating improvement of insulin resistance syndrome such as hyperinsulinemia caused by metabolic disorder.

Thus, the present invention has developed the therapeutic effect of the pure compound dehydroeburicoic acid (TT) of the extract from Antrodia camphorata for type 2 diabetes and dyslipidemia. After the mice with type 2 diabetes induced with high-fat-diet are orally administered with the pure compound dehydroeburicoic acid (TT), not only blood glucose and insulin levels are significantly decreased, but also plasma triacylglycerol and total cholesterol are decreased. TT significantly increases membrane expressions levels of GLUT4 in skeletal muscle to improve glucose uptake. In addition, through administration of TT to HFD-induced diabetic mice, phosphorylation of AMP-activated protein kinase (AMPK) in skeletal muscle and liver tissue is increased, and the efficacy of AMPK phosphorylation is positively correlated with the feeding amount of TT. Furthermore, administration of TT can inhibit liver glucose production and is associated with decrease in mRNA expression of G6 Pase. Administration of TT to diabetic mice results in decrease in blood glucose level through both enhancing GLUT4 membrane protein in skeletal muscle and reducing liver glucose production. Administration of TT can reduce mRNA expression of 11β-HSD1 in liver, resulting in attenuating insulin resistance. TT promotes fatty acid oxidation through inhibiting FAS in liver lipid production and increasing expression level of PPARα protein in fatty acid oxidation in liver, accompanied with increasing mRNA expression levels of CPT1a and UCP3. In addition, decrease in mRNA expression levels of SREBP1c, aP2, and GAPT in liver can reduce synthesis of triacylglycerol in liver tissues, thereby reducing plasma triacylglycerol and fatty liver. TT reduces protein expression levels of lipid synthesis genes including PPARγ and FAS present in adipose tissue, which possibly facilitates decrease in adipocyte differentiation and lipid storage. Further, TT reduces mRNA expression level of SREBP2, which results in decrease in blood total cholesterol. This study shows that TT has excellent therapeutic potential for conditions related to type 2 diabetes.

While we have shown and described various embodiments in accordance with the present invention, it is clear to those skilled in the art that further embodiments may be made without departing from the scope of the present invention.

What is claimed is:

1. Use of a pure compound extracted from Antrodia camphorata, characterized in that the pure compound comprises dehydroeburicoic acid (C₃₁H₄₈O₃) and is used in the preparation of a pharmaceutical composition for controlling appetite, regulating metabolism of lipids and glucoses in peripheral tissues, alleviating a condition, or treating a disease selected from metabolic syndrome, obesity, fatty liver, hyperinsulinemia, or type 2 diabetes.

2. Use according to claim 1, wherein the pure compound further comprises a methanol(MeOH) extract and Antcin K, and the condition is hyperglycemia.

3. Use according to claim 1, wherein the metabolic syndrome is insulin resistance syndrome.

4. Use according to claim 1, wherein the condition is elevation in liver total lipids.

5. Use according to claim 1, wherein the condition is elevation in liver triacylglycerol.

6. Use according to claim 1, wherein the condition is elevation in serum free fatty acids (FFA).

7. Use according to claim 1, wherein the condition is elevation in visceral fat.

8. Use according to claim 1, wherein the condition is body weight gain.

9. Use according to claim 2, wherein the methanol extract and Antcin K partially act via an insulin-dependent pathway.


SEQUENCE LISTING (20 sequences)

SEQ ID NO: 1 - DNA, 22 bp, Artificial Sequence
  primer
gaacaactaaagcctctgaaac

SEQ ID NO: 2 - DNA, 22 bp, Artificial Sequence
  primer
ttgctcgatacataaaacactc

SEQ ID NO: 3 - DNA, 20 bp, Artificial Sequence
  primer
ggctgttgtctaccataagc

SEQ ID NO: 4 - DNA, 20 bp, Artificial Sequence
  primer
aggaagaaacgtgtcaagaa

SEQ ID NO: 5 - DNA, 18 bp, Artificial Sequence
  primer
cagtcctgaataagaggt

SEQ ID NO: 6 - DNA, 20 bp, Artificial Sequence
  primer
tggacaaagatggcagcaga

SEQ ID NO: 7 - DNA, 20 bp, Artificial Sequence
  primer
cagttttatccctagaagca

SEQ ID NO: 8 - DNA, 19 bp, Artificial Sequence
  primer
tctcacgactcaatagctg

SEQ ID NO: 9 - DNA, 20 bp, Artificial Sequence
  primer
cttgtgaccctactacatcc

SEQ ID NO: 10 - DNA, 20 bp, Artificial Sequence
  primer
tcatagcagaaccttaatcc

SEQ ID NO: 11 - DNA, 20 bp, Artificial Sequence
  primer
atatcattgaaaagcgctac

SEQ ID NO: 12 - DNA, 20 bp, Artificial Sequence
  primer
attttcaagtccacatcact

SEQ ID NO: 13 - DNA, 20 bp, Artificial Sequence
  primer
tcacctggaagacagctcct

SEQ ID NO: 14 - DNA, 19 bp, Artificial Sequence
  primer
tgcctgccactttccttgt

SEQ ID NO: 15 - DNA, 20 bp, Artificial Sequence
  primer
gaggtgactacagccttctg

SEQ ID NO: 16 - DNA, 20 bp, Artificial Sequence
  primer
taggaagtgcttccatgtct

SEQ ID NO: 17 - DNA, 20 bp, Artificial Sequence
  primer
aagcagagcaatggcagcat

SEQ ID NO: 18 - DNA, 21 bp, Artificial Sequence
  primer
gagcaatcataggctgggtca

SEQ ID NO: 19 - DNA, 21 bp, Artificial Sequence
  primer
tctccaccttccagcagatgt

SEQ ID NO: 20 - DNA, 23 bp, Artificial Sequence
  primer
agctcagtaacagtccgcctaga